(19) United States
(12) Patent Application Publication
(10) Pub. No.: US 2003/0023111 A1
(43) Pub. Date: Jan. 30, 2003

(54) Process for the production of 1,2,4-triaminobenzene carbamic acid esters

(76) Inventors: Hans-Joachim Lankau, Weinbohla (DE); Klaus Unverferth, Dresden (DE); Thomas Arnold, Radebeul (DE); Jurgen Schaefer, Radebeul (DE); Peter Meisel, Dresden (DE); Wilfried Thiel, Langebruck (DE)

Correspondence Address: Teresa Stanek Rea, Esquire, BURNS, DOANE, SWECKER & MATHIS, L.L.P., P.O. Box 1404, Alexandria, VA, 22313-1404, US

(21) Appl. No.: 10/201,296

(22) Filed: Jul. 24, 2002

(30) Foreign Application Priority Data
Jul. 25, 2001 (DE) 101 36 046.0

(51) Int. Cl. C07C 269/04

(52) U.S. Cl. 560/27

(57) ABSTRACT

A process for the production of 1,2,4-triaminobenzene carbamic acid esters is described, said process being characterised by the use of pyrocarbonate or polypyrocarbonate. The use of pyrocarbonate or polypyrocarbonate brings about a rise in yield and increased purity of the product.

### FIELD OF THE INVENTION

The present invention relates to a novel process for the production of 1,2,4-triaminobenzenes of the general formula I

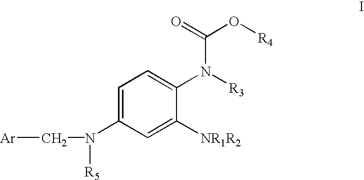

in which the symbols R₁, R₂, R₃, R₄, R₅ and Ar have the following meanings:

R₁, R₃, R₅: hydrogen, C₁-C₆ alkyl, C₁-C₆ alkanoyl or the residue Ar;

R₂: hydrogen or C₁-C₆ alkyl;

R₄: C₁-C₆ alkyl, C₃-C₇ cycloalkyl, C₁-C₆ alkanoyl or the residue Ar;

Ar: phenyl residue, which is substituted by the residues R₆, R₇ and/or R₈, wherein said residues R₆, R₇ and R₈ are identical or different and mean C₁-C₆ alkyl, C₃-C₇ cycloalkyl or halogen; from compounds of the general formula II

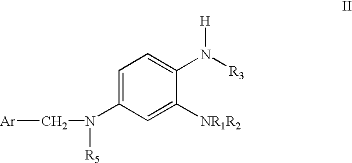

in which R₁, R₂, R₃, R₅ and Ar have the stated meanings by N-acylation with a suitable pyrocarbonate or polypyrocarbonate of the general formula III

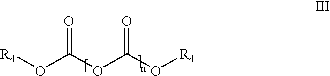

in which n=1, 2, 3 and R₄ has the stated meaning.

Prior art

The production of 1,2,4-triaminobenzene carbamic acid esters is known. For example, the production of 2-carb-ethoxyamino-5-(2,4,6-trimethylbenzylamino)aniline and the hydrochloride thereof as compound 81 is described in the journal Chemiker Zeitung 103 (1979), page 390.

EP 0 554 543 B1 discloses that 1,2,4-triaminobenzene derivatives (for example in the general formula I R₁, R₂, R₃, R₅=H, R₄=C₂H₅, Ar=4-F-phenyl) having an anticonvulsive action are produced by reacting compounds of the general formula II with a chlorocarbonic acid derivative to yield compounds of the general formula I.

It is clear from the description that compounds of the general formula I are generated exclusively using chlorocarbonic acid derivatives in the presence of a basic catalyst. Acylation with chlorocarbonic acid derivatives without a catalyst gives rise to unsatisfactory results. The disadvantage primarily resides in the low selectivity of the reaction.

The catalysts added to increase selectivity, primarily N-ethyldiisopropylamine, form salts with the liberated hydrogen chloride and must be removed after the reaction, so entailing costs.

The product may moreover be contaminated with secondary products, especially those containing chlorine, which are typical of the process and can be removed only with difficulty. Another disadvantage of using chlorocarbonic acid derivatives is that compounds of the general formula II act as acid scavengers and may thus enter to a greater extent into secondary reactions, so resulting in losses of yield of the final product.

There is accordingly a requirement for a process which avoids the stated disadvantages of the prior art and furthermore meets today's more stringent requirements for the environmental compatibility of production processes by avoiding secondary products. It is furthermore advantageous to reduce the number of process steps.

The object of the invention is accordingly to make it possible to obtain compounds of the general formula I with improved yield and purity relative to the known process and to dispense with basic catalysts for the production of compounds of the general formula I.

Description of the invention

The target product is produced according to the invention by using suitable pyrocarbonates or polypyrocarbonates of the general formula III

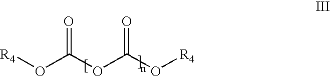

in which n=1, 2 or 3 and R₄=C₁-C₆ alkyl, C₃-C₇ cyclo-alkyl, C₁-C₆ alkanoyl or the residue Ar.

The reaction with pyrocarbonic acid ethyl ester (n=1, R₄=ethyl) has proved particularly advantageous.

According to the invention, the substituted triaminobenzene compound is reacted with a pyrocarbonate or polypyrocarbonate which bears the desired residue R₄. Since gaseous CO₂ arises and escapes during the reaction, it is possible to dispense with an acid scavenger. Even at low temperatures, the triaminobenzene derivatives react with pyrocarbonic acid esters on the desired nitrogen atom to yield the corresponding carbamates. Addition of a catalyst is not necessary. The carbamate usually crystallises out in very pure form during the reaction. The reaction is very readily controllable by observing the escaping carbon dioxide. The end point of the reaction is reached once the escape of carbon dioxide has ceased.

The process and the compounds produced using the process according to the invention unexpectedly exhibit the following advantages:

1. The reaction of the triaminobenzene derivative of the formula II with pyrocarbonate or polypyrocarbonate results in a distinct increase in yield.

2. Distinctly fewer contaminants are detected in the crude product.

3. The selectivity of the reaction is very high.

4. Use of a catalyst is unnecessary.

5. The pyrocarbonic acid ethyl ester process is very readily controllable as conversion may be monitored continuously by the CO₂ which is formed.

6. The improved purity of the crude product cuts out recrystallisation stages.

One major advantage of the process is dispensing with the addition of the catalysts usable according to the prior art. These catalysts are a burden upon the process, which is avoided by the process according to the invention.

The following examples illustrate the invention.

### Exemplary embodiment 1:

41.3 g of pyrocarbonic acid ethyl ester were added dropwise at 5-7° C. within 1 hour to an ethanolic solution comprising 57.8 g (0.25 mol) of 4-(4-fluorobenzylamino)-1,2-phenylenediamine. The suspension was stirred for a further 3 hours at 20-21° C., cooled and stirred for 2 hours at 2-5° C. The substance was removed by suction filtration and washed four times with approx. 30 ml portions of cold ethanol. Yield: 61.2 g (calc. dry), corresponding to 81% of theoretical.

### Comparative Example 1:

42.6 g of N-ethyldiisopropylamine were added briskly at 3-9° C. and 33.1 g of ethyl chloroformate were added dropwise at 8-9° C. within 1.25 hours to an ethanolic solution comprising 53.2 g (0.23 mol) of 4-(4-fluorobenzylamino)-1,2-phenylenediamine. The suspension was stirred for a further 3 hours at 20-21° C., cooled and stirred for 2.5 hours at 2-5° C. The substance was removed by suction filtration and washed four times with approx. 25 ml portions of cold ethanol. Yield: 29.7 g (calc. dry), corresponding to 43% of theoretical.

### Exemplary embodiment 2:

1.98 kg of pyrocarbonic acid ethyl ester were added dropwise at 6-11° C. within 2.5 hours to 21.45 kg of an ethanolic solution comprising 2.78 kg (12 mol) of 4-(4-fluorobenzylamino)-1,2-phenylenediamine. The suspension was stirred for a further 3 hours, cooled and stirred for 2 hours at 5 to −5° C. The substance was removed by suction filtration and washed five times with 1 litre portions of cold ethanol. Yield: 2.4 kg (calc. dry), corresponding to 67% of theoretical.

### Comparative Example 2:

188 kg of N-ethyldiisopropylamine were added briskly at 8° C. and 145.5 kg of ethyl chloroformate were run in at 8-15° C. within 3.25 hours to an ethanolic solution comprising 303.1 kg (1.31 kmol) of 4-(4-fluorobenzylamino)-1,2-phenylenediamine. The suspension was stirred for a further 3 hours at 19-23° C., cooled and stirred for 4 hours at 1-5° C. The substance was removed by centrifugation and washed with a total of 220 litres of cold ethanol. Yield: 186 kg (calc. dry), corresponding to 46% of theoretical.

The purity of the crude product (general formula I R₁, R₂, R₃, R₅=H, R₄=C₂H₅) produced using the process according to the invention and using the original process was determined by means of HPLC diagrams. The HPLC diagrams demonstrate that the crude product of the process according to the invention is distinctly purer. [image] Diagram of the crude product of the general formula I with R₁, R₂, R₃, R₅=H and R₄=C₂H₅ (produced using the process according to the invention). [image] Diagram of the crude product of the general formula I with R₁, R₂, R₃, R₅=H and R₄=C₂H₅ (produced using the original process).

1. A process for the production of 1,2,4-triaminobenzenes of the general formula I 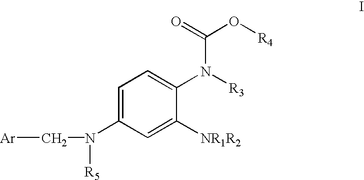
in which the symbols R₁, R₂, R₃, R₄, R₅ and Ar have the following meanings:
R₁, R₃, R₅: hydrogen, C₁-C₆ alkyl, C₁-C₆ alkanoyl or the residue Ar;
R₂: hydrogen or C₁-C₆ alkyl;
R₄: C₁-C₆ alkyl, C₃-C₇ cycloalkyl, C₁-C₆ alkanoyl or the residue Ar;
Ar: phenyl residue, which is substituted by the residues R₆, R₇ and/or R₈₁ wherein said residues R₆, R₇ and R₈ are identical or different and mean C₁-C₆ alkyl, C₃-C₇ cycloalkyl or halogen; characterised in that compounds of the general formula II 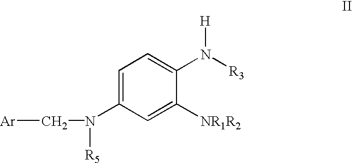
in which R₁, R₂, R₃, R₅ and Ar have the stated meanings, are reacted with a pyrocarbonate or polypyrocarbonate of the general formula III 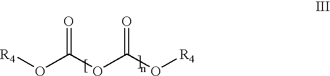
in which n=1, 2, 3 and R₄ has the stated meaning.

2. A process according to claim 1, characterised in that pyrocarbonic acid ethyl ester is preferably used as the pyrocarbonate or polypyrocarbonate.

3. A process according to claims 1 and 2, characterised in that the reaction is carried out at a temperature of −20 to 100° C., preferably of 0° C. to 30° C.

4. A process according to claims 1 to 3, characterised in that the reaction is preferably performed in the presence of a solvent.

5. A process according to claims 1 to 3, characterised in that ethanol is used as the solvent.
